(19) United States
(12) Patent Application Publication
(10) Pub. No.: US 2021/0363587 A1
(43) Pub. Date: Nov. 25, 2021

(54) EPIGENETIC HISTONE REGULATION MEDIATED BY CXorf67

(71) Applicant: St. Jude Children's Research Hospital, Memphis, TN (US)

(72) Inventors: David Ellison, Memphis, TN (US); Wilda Orisme, Memphis, TN (US); Ji Wen, Memphis, TN (US)

(73) Assignee: St. Jude Children's Research Hospital, Memphis, TN (US)

(21) Appl. No.: 16/967,993

(22) PCT Filed: Feb. 6, 2019
(86) PCT No.: PCT/IB2019/050968

§ 371 Date: Aug. 6, 2020

Related U.S. Application Data
(60) Provisional application No. 62/627,291, filed on Feb. 7, 2018.

(51) Int. Cl. C12Q 1/6886

(52) U.S. Cl. CPC C12Q 1/6886; C12Q 2600/156; C12Q 2600/158

(57) ABSTRACT

Compositions and methods are provided for modifying the expression or activity of CXorf67 in order to reduce the activity of PRC2. Increased expression of CXorf67 was identified in certain cancers, including PFA ependymomas. Thus, provided herein are methods for reducing PRC2 activity in order to treat cancer. The methods and compositions can be used to treat symptoms cancer or to screen for compounds useful in decreasing PRC2 activity and treating cancer. Further provided are methods of identifying subjects at an increased risk of developing cancer by measuring the expression or activity of CXorf67 or the mutation of specific sites within CXorf67.

### FIELD OF THE INVENTION

The invention relates to the field of cell biology, particularly cancer biology and epigenetics. Specifically, the invention relates to a method for modulating PRC2 activity by administering a modulator of CXorf67 activity for therapeutic or research purposes.

### REFERENCE TO A SEQUENCE LISTING SUBMITTED ELECTRONICALLY AS A TEXT FILE

The instant application contains a Sequence Listing which has been submitted in ASCII format and is hereby incorporated by reference in its entirety. Said ASCII copy, created on Feb. 6, 2019, is named S88435_1190WO_0045_6_SEQLIST.txt, and is 7.25 KB in size.

### BACKGROUND OF THE INVENTION

Epigenetic control of gene expression in cells is mediated in part by modifications to DNA nucleotides including the cytosine methylation status of DNA. It has been known in the art for some time that DNA may be methylated at the 5 position of cytosine nucleotides to form 5-methylcytosine. Methylated DNA in the form of 5-methylcytosine is reported to occur at positions in the DNA sequence where a cytosine nucleotide occurs next to a guanine nucleotide. These positions are termed “CpG” for shorthand. It is reported that more than 70% of CpG positions are methylated in vertebrates (Pennings et al., 2005). Regions of the genome that contain a high proportion of CpG sites are often termed “CpG islands”, and approximately 60% of human gene promoter sequences are associated with such CpG islands (Rodriguez-Paredes and Esteller, 2011). In active genes these CpG islands are generally hypomethylated. Methylation of gene promoter sequences is associated with stable gene inactivation. DNA methylation also commonly occurs in repetitive elements including Alu repetitive elements and long interspersed nucleotide elements (Herranz and Estellar, 2007; Allen et al, 2004).

The involvement of DNA methylation in cancer was reported as early as 1983 (Feinberg and Vogelstein, 1983). DNA methylation patterns observed in cancer cells differ from those of healthy cells. Repetitive elements, particularly around pericentromeric areas, are reported to be hypomethylated in cancer relative to healthy cells but promoters of specific genes have been reported to be hypermethylated in cancer. The balance of these two effects is reported to result in global DNA hypomethylation in cancer cells (Rodriguez-Paredes; Esteller, 2007). Polycomb group (PcG) proteins are chromatin modifying enzymes that are dysregulated in many human cancers. Histone H3 is one of the five main histone proteins involved in the structure of chromatin in eukaryotic cells. Featuring a main globular domain and a long N-terminal tail, H3 is involved with the structure of the nucleosomes. Histone proteins are post-translationally modified; however, histone H3 is the most extensively modified of the five histones. Histone H3 is an important protein in the emerging field of epigenetics, where its sequence variants and variable modification states are thought to play a role in the dynamic and long term regulation of genes.

The Polycomb Repressive Complex 2 (PRC2), which includes SUZ12 (suppressor of zeste 12), EED (embryonic ectoderm development) and the catalytic subunit, EZH2 (enhancer of zeste homolog 2), modulates gene expression by methylating the core histone H3 lysine 27 (H3K27me3) at and around the regulatory region, such as the promoter promoter of target genes. PRC2 is one critical component of cellular machinery involved in the epigenetic regulation of gene transcription and plays critical functions in tissue development and differentiation and in regeneration. Although EZH2 is the catalytic subunit, PRC2 requires at least EED and SUZ12 for its methyltransferase activity. EED, SUZ12 and EZH2 are dysfunctional in many cancers, including but not limited to breast cancer, prostate cancer, hepatocellular carcinoma. EZH2 activating mutations have been identified in DLBCL (diffused large B cell lymphoma) patients and FL (follicular lymphoma) patients. Inhibition of PRC2 methyltransferase activity by compounds competing with the cofactor S-adenosyl methionine (SAM) in DLBCL reverses H3K27 methylation, re-activates expression of target genes and inhibits tumor growth/proliferation. Therefore, PRC2 provides a pharmacological target for DLBCL and other cancers in which its function is dysregulated.

Ependymomas are neuroepithelial tumors of the central nervous system (CNS), presenting in both adults and children but accounting for almost 10% of all pediatric CNS tumors and up to 30% of those in children under 3 years (Bouffet et al., 2009; McGuire et al., 2009; Rodriguez et al., 2009). In children, most ependymomas arise in the posterior fossa, while most adult ependymomas present around the lower spinal cord and spinal nerve roots. Ependymomas display a wide range of morphological features, and several variants are listed in the World Health Organization (WHO) classification (Ellison et al., 2016). These variants are assigned to three WHO grades (I-III), but the clinical utility of this classification is acknowledged to be limited (Ellison et al., 2011). Posterior fossa (PF) type A (PFA) tumors are found mainly in infants and young children (median age 3 yrs) and have a relatively poor outcome, while posterior fossa type B (PFB) tumors are generally found in young adults (median age 30 yrs) and are associated with a better prognosis (Pajtler et al., 2015; Witt et al., 2011). PFA tumors show few copy number alterations (CNAs), while PFB tumors harbor multiple CNAs that tend to affect entire chromosome arms. While recurrent structural variants (SVs) are found in ST ependymomas, recurrent SVs or other mutations, such as single nucleotide variants (SNVs) and insertions or deletions (indels), have not so far been reported in PF ependymomas (Mack et al., 2014; Parker et al., 2014). PFA and PFB ependymomas also differ with respect to H3K27 trimethylation (H3K27-me3) status; there is a global reduction in the H3K27 trimethylation in PFA tumors (Panwalkar et al., 2017). Immunohistochemical analysis of H3K27me3 demonstrates global reduction PFA ependymoma, and this biomarker is a powerful predictor of outcome. H3K27-me3 status can be modulated by PRC2 activity. The present application reports that, in PF ependymomas, PRC2 activity is regulated by CXorf67, the protein product of a novel gene of previously unknown function, which is overexpressed in PFA ependymomas.

### SUMMARY OF THE INVENTION

Compositions and methods are provided for modifying the expression or activity of CXorf67 in order to alter the activity of PRC2 and its miscellaneous downstream effects. Increased expression of CXorf67 has been identified in certain cancers, including PFA ependymomas. Thus, provided herein are methods for altering PRC2 activity in order to treat cancer or other diseases where the interaction between CXorf67 and PRC2 might be involved in pathogenesis. The methods and compositions can be used to treat symptoms of cancer or to screen for compounds useful in decreasing PRC2 activity and treating cancer. Further provided are methods of identifying subjects at an increased risk of developing cancer by measuring the expression or activity of CXorf67 or the mutation of specific sites within CXorf67.

### BRIEF DESCRIPTION OF THE FIGURES

FIG. 1 shows expression of CXorf67 in CNS tumors. Elevated levels of CXorf67 are seen in PFA ependymomas and CNS germ cell tumors, among which the germinoma is known to overexpress CXorf67. Data derived from published gene expression profiling datasets.

FIG. 2 demonstrates that overexpression of CXorf67 is associated with CXorf67 promoter region hypomethylation in PFA ependymomas, in contrast to PFB or supratentorial (ST) ependymomas. Each cell in the heatmap and the black lines leading from them represent CpG islands and the lines are drawn to the corresponding position in relation to the gene itself (blue bar with arrowheads). The promoter region is identified by yellow bars both (i) above the cells in the heatmap and (ii) in the 5′ UTR to the left of the gene. Relative hypomethylation is gray/blue and the cells in the heatmap that correspond to the promoter region of CXorf67 (yellow bar below) in PFA tumors (black arrow to the right of the heatmap) show an increased number of white or blue signals compared to the red hypermethylation signals in the promoter region of PFB and ST tumors.

FIG. 3 shows sections through two ependymomas. (A.) is a PFA ependymoma and shows positive staining of tumor cell nuclei where the immunohistochemical method has detected the expression of CXorf67. (B.) is a PFB ependymoma, in which there is no expression of CXorf67 and therefore no positive staining of tumor cells.

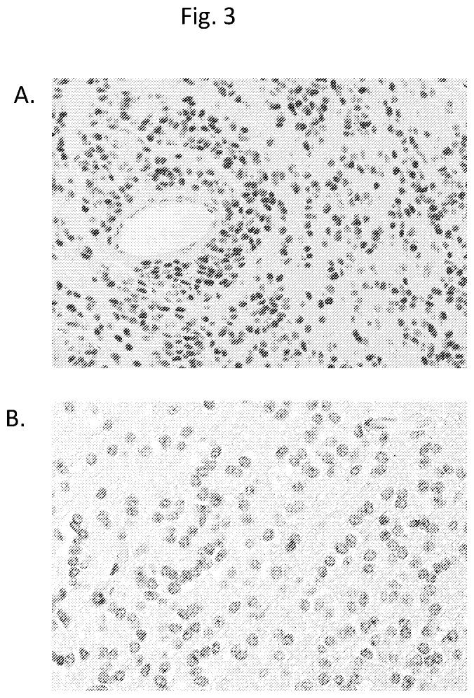

FIG. 4 presents the immunoprecipitation-mass spectrometry (IP-MS) results for CXorf67 immunoprecipitation in the Daoy cell line, which overexpresses CXorf67. An anti-CXorf67 antibody was shown to pull down EZH2, SUZ12, and EED, the three core components of PRC2, and other elements of the complex and other proteins with different functions.

FIG. 5 presents a volcano plot of the IP-MS results of FIG. 4, highlighting components of PRC2.

FIG. 6 presents a SAINT plot (A) and corresponding data (B) based on IP-MS results for CXorf67 IP/MS in both Daoy and U2-OS cell lines. Common elements are components of PRC2.

FIG. 7 shows a compilation of western blots in tabular form. The antibody used for IP is indicated above each column. The antibody used for the blot is indicated beside each row. Input refers to the total protein after pre-clearing; SN refers to the supernatant after antibody-total protein binding; and E refers to the elution of the protein binding complex.

FIG. 8 shows by dual-antibody immunofluorescence the effects of expressing CXorf67 in a cell line (HEK293T) that normally lacks expression of CXorf67. H3K27-me3 (red color) is downregulated in cells that have been transfected with CXorf67 (green color).

FIG. 9 presents two western blots, which demonstrate the effects on H3K27-me3 of (A.) transfecting human neural stem cells (hNSC) with CXorf67 and (B.) knocking down CXorf67 in the Daoy cell line. hNSCs do not express CXorf67 and have high levels of H3K27-me3, while Daoy cells express CXorf67 and negligible H3K27-me3. In both situations, altering CXorf67 levels had the anticipated reciprocal effect on H3K27-me3.

FIG. 10 shows CXorf67 mutations discovered in 22 PFA ependymomas.

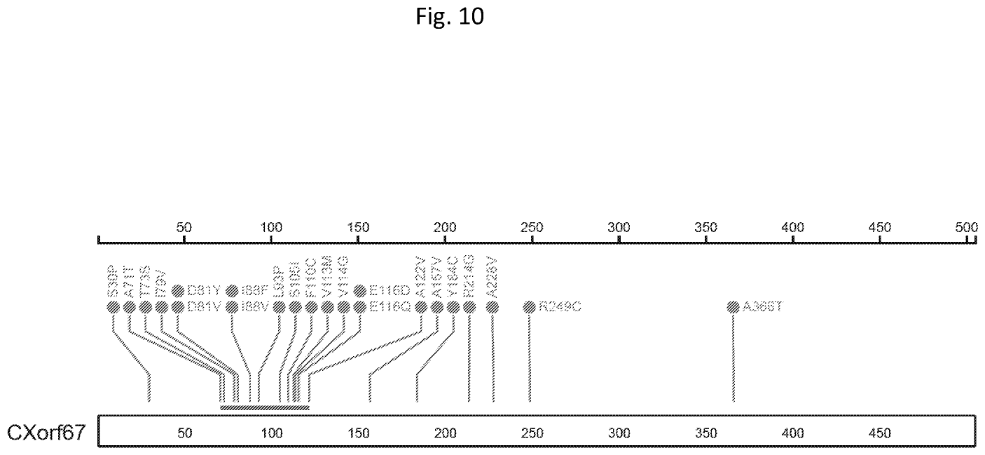

FIG. 11 shows expression of murine CXorf67 in NIH3T3 cells.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### 1. Overview

It is increasingly appreciated that (i) histone modifications are important in the regulation of gene expression and that (ii) PRC2, one of the polycomb group (PcG) proteins, is in turn an important regulator of histone modifications. PRC2 can function in modulating gene expression at key moments during tissue development, e.g. it has a well-known role in the inactivation of one X chromosome. A better understanding of such functions is important for a range of diseases, including cancer, where epigenetic regulation and, specifically, histone modifications, are pathologically altered.

The compositions and methods of the instant claims are useful in modifying the activity of polycomb repressive complex 2 (PRC2) by modulating the activity of CXorf67. As used herein, “PRC2” refers to any complex including SUZ12 (suppressor of zeste 12), EED (embryonic ectoderm development) and the catalytic subunit, EZH2 (enhancer of zeste homolog 2). Other active components and co-factors of PRC2 include JARID2, AEBP2, PCL, SET, PHF1, RBBP7/4 (RbAp46/48), and PCL1-3. Accordingly, components of the PRC2 include, but are not limited to, SUZ12, EED, and EZH2, or any combination thereof, along with JARID2, AEBP2, PCL, SET, PHF1, RBBP7/4, and PCL1-3. PRC2 can regulate activity of genes by methylating a histone at or near regions known to be sites of target gene transcriptional regulation. Accordingly, “PRC2 activity” or the “activity of PRC2” refers to the ability to methylate residues of a histone, particularly lysine 27 on histone H3. For example, PRC2 activity can refer to histone methyltransferase activity. In some embodiments, PRC2 activity refers to the ability to methylate the core histone H3 lysine 27 (H3K27me3) at and around the promoter regions of target genes. Thus, PRC2 can regulate gene transcription by epigenetic regulation of the promoter region of target genes.

The Polycomb group (PcG) proteins form chromatin-modifying complexes that are essential for embryonic development and stem cell renewal and are commonly deregulated in cancer. The target genes of PcG regulation have been studied and identified using genome-wide location analysis in human embryonic and developing tissues. For genes activated during differentiation, PcGs are displaced. However, for genes repressed during differentiation, the genes are already bound by the PcGs in nondifferentiated cells despite being actively transcribed. Thus PcGs could be part of a preprogrammed memory system established during embryogenesis marking certain key genes for repressive signals during subsequent developmental and differentiation processes. Accordingly, modulating PRC2 activity can modulate the expression or activity of genes involved in embryogenesis, developmental, and differentiation processes. See, for example, Bracken, A. P., et al. (2006) Genes Dev 20(9): 1123-1136, herein incorporated by reference.

The nucleic acid molecule, “CXorf67” encodes a nucleic acid or protein product that is primarily located in the nucleus of the cell and modulates PRC2 activity. The CXorf67 gene or CXorf67 protein can also be referred to as “EZH2 inhibiting protein” or “EZHIP”. CXorf67 is overexpressed in tumors such as ependymomas and germinomas and can modulate PRC2 activity by binding to any single component of PRC2 or a combination of PRC2 components. In some embodiments, CXorf67 is overexpressed in tumors and can modulate molecules that affect cellular functions, such as epigenetic regulation. In some embodiments, PRC2 can bind SUZ12, EED, and EZH2 and modulate PRC2 activity. CXorf67 refers to the nucleic acid sequence set forth in SEQ ID NO: 1, or variants thereof, and the amino acid sequence of CXorf67 is set forth in SEQ ID NO: 2, or active variants thereof. CXorf67 is a single exon gene of unknown function. Its protein product is predicted to be ‘disordered’, apart from one region towards the N terminus.

### 2. Methods of Modulating PRC2 Activity

Compositions and methods are provided herein for modulating activity of PRC2 by modulating the expression or activity of CXorf67. Modulating the activity of PRC2 refers to increasing or decreasing PRC2 activity relative to an appropriate control. Likewise, modulating the expression or activity of CXorf67 refers to increasing or decreasing CXorf67 expression or activity relative to an appropriate control. PRC2 activity can be measured, for example, by measuring the methylation status or methylation level of any histone marker associated with PRC2. For example, the complex has histone methyltransferase activity and PRC2 activity can be determined by measuring the histone methyltransferase activity. In some embodiments, PRC2 activity can be determined by measuring the methylation status or methylation level of histone H3. In specific embodiments, PRC2 activity can be determined by measuring the methylation status or methylation level of lysine 27 on histone H3 (H3K27). For example, in particular embodiments, PRC2 activity produces trimethylated lysine 27 on histone H3 (H3K27me3). Mammalian cells have several known sequence variants of histone H3. These are denoted as Histone H3.1, Histone H3.2, Histone H3.3, Histone H3.4 (H3T), Histone H3.5, Histone H3.X and Histone H3.Y but have highly conserved sequences differing only by a few amino acids. As used herein, “histone H3” or “H3” refers to any variant of histone H3. In particular embodiments, reducing the expression or activity of CXorf67 can alter the methylation status of H3K27 from tri-methylated to di-methylated, or from tri-methylated to mono-methylated, or from di-methylated to mono-methylated. In some embodiments, reducing the expression or activity of CXorf67 can alter the methylation status of any other Histone, such as any histone H3. As used herein, genes encoding histone H3 proteins can include HIST1H3A, HIST1H3B, HIST1H3C, HIST1H3D, HIST1H3E, HIST1H3F, HIST1H3G, HIST1H3H, HIST1H3I, HIST1H3J, HIST2H3A, HIST2H3C, HIST2H3D, H3F3A, and/or H3F3B. In specific embodiments reducing the expression or activity of CXorf67 can remove the methylation from H3K27 or be associated instead with the acetylation of H3K27.

The methylation status of histone H3 can be measured at multiple locations. In specific embodiments, the methylation status of histone H3 can be measured upstream of a target gene in a mammalian chromosome. For example, the methylation status of histone H3 can be measured about 10 bp, 20 bp, 30 bp, 40 bp, 50 bp, 60 bp, 70 bp, 80 bp, 90 bp, 100 bp, 150 bp, 200 bp, 250 bp, 300 bp, 400 bp, 500 bp, 600 bp, 700 bp, 800 bp, 1000 bp, or more base pairs upstream (5′) of a target gene. The methylation status of histone H3 can be measured in the regulatory regions of any target gene of interest. Regulatory regions can include any promoter, enhancer, super enhancer, long non-coding RNA (lncRNA), or repressor associated with a target gene. In some embodiments, the methylation status of Histone H3 can be measured at the lysine at position 27 when the histone is at or near the promoter of any gene of interest. As used herein, the histone is near a promoter or enhancer of interest when the histone is within 5 bp, 10 bp, 15 bp, 20 bp, 25 bp, 30 bp, 35 bp, 40 bp, 45 bp, 50 bp, 75 bp, or 100 bp of the promoter. at or near the promoter of a target gene.

In specific embodiments, modulating the expression or activity of CXorf67 can alter the methylation status of a histone. As used herein, “methylation status” or “methylation level” can refer to histone methylation or methylation of histone tails. Histone methylation can be measured on any histone disclosed herein, such as Histone H3. In specific embodiments, “methylation status”, “methylation level”, or “histone methylation” refers to methylation of residues on histone tails, particularly at K27 of histone H3, which is encoded by multiple genes, including H3F3A and those in the HIST1 cluster located on 6p22.2 (26216000-2628500), that is to say the number of CH3 group(s) on the lysine 27 of Histone H3. According to the invention, the histone methylation on H3K27 can be a mono-methylation, di-methylation or a tri-methylation. In specific embodiments, altering the expression or activity of CXorf67 can alter the histone methylation of H3K27 from tri-methylation to di-methylation, from tri-methylation to mono-methylation, or from di-methylation to mono-methylation.

In certain embodiments, “methylation status”, “methylation level”, or “histone methylation” refers to methylation of any histone. In some embodiments, the methylation level of any Histone 3 can be measured to determine methylation status. For example, the methylation at any appropriate position of HIST1H3A, HIST1H3B, HIST1H3C, HIST1H3D, HIST1H3E, HIST1H3F, HIST1H3G, HIST1H3H, HIST1H3I, HIST1H3J, HIST2H3A, HIST2H3C, HIST2H3D, H3F3A, and/or H3F3B can be measured to determine the methylation status and PRC2 activity.

Methods for extracting chromatin from biological samples and determining the histone methylation level are well known in the art. Commonly, chromatin isolation procedures comprise lysis of cells after one step of crosslink that will fix proteins that are associated with DNA. After cell lysis, chromatin can be fragmented, immunoprecipitated and DNA can be recovered. DNA can then be extracted with phenol, precipitated in alcohol, and dissolved in an aqueous solution. The H3K27 methylation level can be determined by chromatin IP (see for example Boukarabila H., et al, 2009) ChIP-chip or by ChIP-qPCR (see for example the materiel and methods part and Wu J. et al., 2006). As used herein, a “control histone methylation value” is the histone methylation level of H3K27 in the HIST1 cluster, or other Histones disclosed herein, determined in a biological sample of a subject not afflicted by a cancer or other cell-proliferative disorder. In specific embodiments, a control or normal level of histone methylation is assessed in a control sample (e.g., sample from a healthy patient, which is not afflicted by a cancer). In some embodiments, the control level of histone methylation refers to the average histone methylation level of H3K27 from several control samples.

The term “methylation status” or “methylation level” refers to the presence, absence, and/or quantity of methylation at a particular codon, nucleotide, or nucleotides within a portion of DNA. The methylation status of a particular DNA sequence (e.g., a DNA biomarker or DNA region as described herein) can indicate the methylation state of every base in the sequence or can indicate the methylation state of a subset of the base pairs (e.g., of a particular codon, of cytosines, or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value” or “methylation level.” A methylation value or level can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, represents the methylation status and can thus be used as a quantitative indicator of methylation status. This is of particular use when it is desirable to compare the methylation status of a sequence in a sample to a threshold value. A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). In specific embodiments, methylation status can be determined as mono-, di-, or tri-methylated. PRC2 activity can refer to the ability of the complex to tri-methylate histone H3 at lysine 27 at or near the promoter of any gene of interest.

In some embodiments, PRC2 activity can be determined by measuring the acetylation status or acetylation level of histone H3. Acetylation has the effect of changing the overall charge of the histone tail from positive to neutral. Thus, in particular embodiments, reducing the expression or activity of CXorf67 can alter the acetylation status of H3K27 from acetylated to de-acetylated or from de-acetylated to acetylated. Acetylation status can be determined by any means known in the art. For example, acetylation can be measured by determining if the lysine residues within the N-terminal tail protruding from the histone core of the nucleosome are acetylated and deacetylated.

Modulating the activity of CXorf67 refers to increasing or decreasing expression of CXorf67 relative to an appropriate control. As used herein, the term “increased” refers to any increase in the expression or activity of CXorf67or PRC2 when compared to the corresponding expression or activity of CXorf67or PRC2 in a control cell. Such an increase may be up to 5%, 10%, 20%, 30%, 40%, 50%, 60%, 70%, 80%, 90%, 95%, or up to 100%, 200%, 300%, 400%, or 500%, or more when compared to an appropriate control.

As used herein, the term “decreased” or “reduced” refers to any reduction in the expression or activity of CXorf67 or PRC2 when compared to the corresponding expression or activity of CXorf67or PRC2 in a control cell. Such a reduction may be up to 5%, 10%, 20%, 30%, 40%, 50%, 60%, 70%, 80%, 90%, 95%, or up to 100% when compared to an appropriate control. Accordingly, the term “reduced” encompasses both a partial knockdown and a complete knockdown of the expression of CXorf67 and PRC2. Thus, in some embodiments, a cell having a lower level of CXorf67 expression compared to an appropriate control level of CXorf67 expression has a level of CXorf67 expression that is at least 5%, 10%, 20%, 30%, 40%, 50%, 60%, 70%, 80%, 90%, 95%, or 100%, lower than an appropriate control level of CXorf67 expression. A level of CXorf67 expression may be determined using any suitable assay known in the art (see, e.g., Molecular Cloning: A Laboratory Manual, J. Sambrook, et al., eds., Third Edition, Cold Spring Harbor Laboratory Press, Cold Spring Harbor, N.Y., 2001; Current Protocols in Molecular Biology, Current Edition, John Wiley & Sons, Inc., New York; and Current Protocols in Protein Production, Purification, and Analysis, Current Edition, John Wiley & Sons, Inc., New York). The CXorf67 expression level may be an mRNA level or a protein level. The sequences of CXorf67 DNA and protein sequences are provided herein as (SEQ ID NO: 1 and 2, respectively) and can be used to design suitable reagents and assays for measuring CXorf67 expression level. Likewise, a cell having a lower level of PRC2 activity compared to an appropriate control level of PRC2 activity has a level of PRC2 activity that is at least 5%, 10%, 20%, 30%, 40%, 50%, 60%, 70%, 80%, 90%, 100%, 200%, 300%, 400%, 500% or lower than an appropriate control level of PRC2 activity.

The terms “measuring” and “determining” are used interchangeably throughout, and refer to methods which include obtaining a subject sample and/or detecting the methylation status or activity of CXorf67 in a sample. In specific embodiments, detecting the methylation status of CXorf67 refers to measuring the methylation status of the promoter of CXorf67 in a sample obtained from an ependymoma. In one embodiment, the terms refer to obtaining a subject sample and detecting the methylation status or expression level of CXorf67 in the sample. In another embodiment, the terms “measuring” and “determining” mean detecting the methylation status or level of H3K27 in a sample. Measuring can be accomplished by methods known in the art and those further described herein including, but not limited to, quantitative polymerase chain reaction (PCR). The term “measuring” is also used interchangeably throughout with the term “detecting” and “determining.”

As used herein the term “sample” or “biological sample” in the context of the present disclosure is a biological sample isolated from a subject and can include, by way of example and not limitation, bodily fluids and/or tissue extracts such as homogenates or solubilized tissue obtained from a subject. Tissue extracts are obtained routinely from tissue biopsy and autopsy material. Bodily fluids useful in the present invention include blood, bone marrow aspirate, urine, saliva or any other bodily secretion or derivative thereof. As used herein “blood” includes whole blood, plasma, serum, circulating cells, constituents, or any derivative of blood. In a particular embodiment, the biological sample is a blood sample, more particularly a biological sample comprising circulating white blood cells (WBC).

Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample”. In a particular embodiment, the sample includes nucleic acids. In specific embodiments, a sample used for measurement of histone methylation level, PRC2 activity, and/or CXorf67 expression or activity is a biological sample comprising nucleic acids.

In some embodiments, an appropriate control level of CXorf67 expression or PRC2 activity may be, e.g., a level of CXorf67 expression or PRC2 activity in a cell, tissue or fluid obtained from a healthy subject or population of healthy subjects. As used herein, a healthy subject is a subject that is apparently free of disease and has no history of disease, e.g., no history of cancer. In some embodiments, an appropriate control level is a level of CXorf67 expression or PRC2 activity in a germ cell from a subject that does not have cancer or a level of PRC2 expression in a population of germ cells from a population of subjects that do not have cancer. In some embodiments, the subject or population of subjects that do not have ependymoma or germinoma are subjects that have a CXorf67 gene locus that contains less than 5, less than 4, less than 3, less than 2, or less than 1 mutation compared to the wild type CXorf67 sequence. The mutation can be a substitution, addition or deletion anywhere in the CXorf67 nucleotide sequence. In specific embodiments, the subject does not have a mutation between codon 71 and 122 of the CXorf67 polynucleotide set forth in SEQ ID NO: 1. In some embodiments, the subject does not have a mutation at position 30, 71, 73, 79, 81, 88, 93, 105, 110, 113, 114, 116, 122, 157, 184, 214, 228, 249, and/or 366 of the CXorf67 polynucleotide set forth in SEQ ID NO: 1. Further mutations in CXorf67 can be identified from the COSMIC and CLINVAR databases set described elsewhere herein. For example the control cell can be from a subject without a mutation in at least one of codons 81, 88, or 116 of the CXorf67 polynucleotide.

In some embodiments, an appropriate control level of CXorf67 expression may be a predetermined level or value, such that a control level need not be measured every time. The predetermined level or value can take a variety of forms. It can be single cut-off value, such as a median or mean. The value can be established based upon comparative groups, such as where one defined group is known to have an ependymoma or germinoma and another defined group is known to not have an ependymoma or germinoma.

Fragments and variants of the CXorf67 polynucleotides and CXorf67 amino acid sequences encoded thereby are encompassed herein. By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein. Biologically active variants of a native polypeptide will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a protein of the invention may differ from that protein by as few as 1-15 amino acid residues, as few as 1-10, such as 6-10, as few as 5, as few as 4, 3, 2, or even 1 amino acid residue. Variant CXorf67 sequences can retain the ability to bind the PRC2 complex.

Variant sequences may also be identified by analysis of existing databases of sequenced genomes. In this manner, corresponding sequences can be identified and used in the methods of the invention.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244; Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990)J Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the website at www.ncbi.nlm.nih.gov. Alignment may also be performed manually by inspection.

In some embodiments, CXorf67 harbors a mutation that can decrease or increase activity of the corresponding protein, or correlate to increased or decreased risk or incidence of an ependymoma or germinoma, particularly a PFA ependymoma. The mutation can be a missense or nonsense mutation.

In specific embodiments, the methods disclosed herein relate to modulating the expression of CXorf67 in order to reciprocally modulate the methylation of H3K27. In some embodiments, the methods disclosed herein relate to reducing the expression of CXorf67 in order to reduce the activity of PRC2. For example, a modulator of CXorf67 can be contacted within a cell in order to reduce the PRC2 activity of the cell. In specific embodiments, the modulator of CXorf67 reduces expression or activity of CXorf67 in order to reduce the activity of PRC2. Accordingly, an inhibitor of CXorf67 is any modulator of CXorf67 that reduces the expression or activity of CXorf67. Thus, by reducing the expression and/or activity of CXorf67, the activity of PRC2 can be reduced and methylation of H3K27 can be reduced or prevented from future methylation.

Reduction (i.e., decreasing) of the expression of a gene (e.g., CXorf67) related to increased the activity of PRC2 and/or methylation of H3K27 can be achieved by any means known in the art. For example, gene expression can be altered by a mutation. The mutation can be an insertion, a deletion, a substitution or a combination thereof, provided that the mutation leads to a decrease in the expression of CXorf67. In specific embodiments recombinant DNA technology can be used to introduce a mutation into a specific site on the chromosome. Such a mutation may be an insertion, a deletion, a replacement of one nucleotide by another one or a combination thereof, as long as the mutated gene leads to a decrease in the expression of CXorf67. Such a mutation can be made by deletion of a number of base pairs. In one embodiment, the deletion of one single base pair could render CXorf67 non-functional, thereby reducing PRC2 activity and, in some embodiments, decreasing methylation status of H3K27 at or near the promoter region of a gene of interest. In other embodiments, multiple base pairs are removed e.g. about 100 base pairs. In still other embodiments, the length of the entire CXorf67 gene is deleted. Mutations introducing a stop-codon in the open reading frame, or mutations causing a frame-shift in the open reading frame could be used to reduce the expression of CXorf67.

Other techniques for decreasing the expression of CXorf67 are well-known in the art. For example, techniques may include modification of the gene by site-directed mutagenesis, restriction enzyme digestion followed by re-ligation, PCR-based mutagenesis techniques, allelic exchange, allelic replacement, RNA interference, or post-translational modification. Standard recombinant DNA techniques such as cloning the CXorf67 gene, digestion of the gene with a restriction enzyme, followed by endonuclease treatment, re-ligation, and homologous recombination are all known in the art and described in Maniatis/Sambrook (Sambrook, J. et al. Molecular cloning: a laboratory manual. ISBN 0-87969-309-6). Site-directed mutations can be made by means of in vitro site directed mutagenesis using methods well known in the art.

In some embodiments the expression of CXorf67 is reduced using interfering nucleic acids or polypeptides. For example, RNA interference or interfering RNAs (“RNAi”) can be used to decrease the expression of a gene responsible for methylation of DNA. “RNAi” refers to a series of related techniques to reduce the expression of genes (see, for example, U.S. Pat. No. 6,506,559, herein incorporated by reference in its entirety). Older techniques referred to by other names are now thought to rely on the same mechanism, but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced. The activity of genes responsible for methylation of DNA as disclosed herein can be reduced using RNA interference including microRNAs and siRNAs. CXorf67 protein expression can be reduced by using RNA interference such as siRNA or shRNA, by using antisense RNA, or by knocking out the gene encoding the CXorf67 protein. In particular embodiments, protein expression or activity of CXorf67 can be reduced using an antisense nucleic acid, a ribozyme, a peptide, an antibody, an antagonist, an aptamer, or a peptidomimetic that reduces the expression or activity of a CXorf67 protein, respectively.

By “reduces” or “reducing” gene expression is intended to mean, the polynucleotide or polypeptide level of CXorf67 is statistically lower than the polynucleotide level or polypeptide level of the same target sequence in an appropriate control or the CXorf67 activity of the cell is statistically lower than the CXorf67 activity of an appropriate control cell. In particular embodiments, reducing the expression of a gene according to the presently disclosed subject matter results in at least a 95% decrease, at least a 90% decrease, at least a 80% decrease, at least a 70% decrease, at least a 60% decrease, at least a 50% decrease, at least a 40% decrease, at least a 30% decrease, at least a 20% decrease, at least a 10% decrease, or at least a 5% decrease of the gene expression when compared to an appropriate control. In other embodiments, reducing the gene expression results in a decrease of about 3%-15%, 10%-25%, 20% to 35%, 30% to 45%, 40%-55%, 50%-65%, 60%-75%, 70%-90%, 70% to 80%, 70%-85%, 80%-95%, 90%-100% in the gene expression when compared to an appropriate control. In specific embodiments the methylation status or methylation profile of histone H3 is reduced by reducing the expression of CXorf67. In some embodiments PRC2 activity is reduced by reducing the expression of CXorf67. Reducing the methylation status or methylation profile of any histone, refers to at least a 95% decrease, at least a 90% decrease, at least a 80% decrease, at least a 70% decrease, at least a 60% decrease, at least a 50% decrease, at least a 40% decrease, at least a 30% decrease, at least a 20% decrease, at least a 10% decrease, or at least a 5% decrease of the methylation status or methylation profile of a histone or any residue within the histone when compared to an appropriate control. Methods to assay for the level of the gene expression, methylation status, methylation profile, the expression of reduced by reducing the expression of CXorf67, or PRC2 activity are discussed elsewhere herein and known in the art.

### 3. Methods of Treatment

In some aspects, the invention relates to methods for modulating CXorf67 gene expression cells for research purposes. In other aspects, the invention relates to methods for modulating CXorf67 gene expression in cells for therapeutic purposes. Cells can be in vitro, ex vivo, or in vivo (e.g., in a subject who has a disease involving increased expression or activity of CXorf67 or PRC2, such as cancer). Thus, in particular embodiments, “administering” a modulator of CXorf67 expression or activity encompasses administration to a subject disclosed herein and contacting the modulator with a cell or other compound outside of a subject. In specific embodiments, a modulator of CXorf67 activity can be administered to a cell or tissue culture or can be used in a screening assay in order to identify compounds that alter PRC2 and/or CXorf67 activity. Accordingly, the methods disclosed herein are useful in identifying modulators of PRC2 and/or CXorf67 activity. In some embodiments, methods for modulating CXorf67 expression in cells comprise delivering to the cells an oligonucleotide that inhibits expression or activity of CXorf67.

“Treatment” or “treating” as used herein refers to curing, healing, alleviating, relieving, altering, remedying, ameliorating, improving, or affecting the condition or the symptoms of a cancer, a cell proliferative disorder or any other condition wherein the interaction of CXorf67 and PRC2 causes a disease or pathogenic condition in a subject by reducing the expression or activity of CXorf67 or by reducing the PRC2 activity of a cell. As used herein the term “symptom” refers to an indication of disease, illness, injury, or that something is not right in the body.

Symptoms are felt or noticed by the individual experiencing the symptom, but may not easily be noticed by others. Others are defined as non-health-care professionals. Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body. As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

Sometimes cancers start in places where it does not cause any symptoms until the cancer has grown quite large. Pancreatic cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage. Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma. Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease. Along with cancers of the skin, some internal cancers can cause skin signs that can be seen. These changes include the skin looking darker (hyperpigmentation), yellow (jaundice), or red (erythema); itching; or excessive hair growth. In some cases, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could be a symptom of cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer. Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size. Indigestion or trouble swallowing could be symptomatic of cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma. A persistent cough or hoarseness could be indicative of cancer. A cough that does not go away may be a sign of lung cancer. Hoarseness can be a sign of cancer of the larynx (voice box) or thyroid.

New or increasingly strong headaches, blurred vision, vomiting, bilateral Babinski sign, drowsiness, impaction/constipation, back flexibility, loss of balance, confusion, and seizures could be symptoms of a cancer of the brain, such as an ependymoma. Likewise, hydrocephalus, headache, vomiting, fatigue, behavior or cognitive changes, ataxia, balance issues, or vision changes can be symptoms of germ cell brain tumors, such as germinomas. Tumors in the suprasellar region of the brain can cause early or delayed puberty, stunted growth, and/or vision problems.

While the signs and symptoms listed above are the more common ones seen with cancer, there are many others that are less common and are not listed here. However, all art-recognized signs and symptoms of cancer are contemplated and encompassed by the instant invention.

In specific embodiments, treatment or treating encompasses a reduction in the size of a tumor disclosed herein. Tumor size can be determined using a variety of methods known in the art, such as, for example, by measuring the dimensions of tumor(s) upon removal from the subject, e.g., using calipers, or while in the body using imaging techniques, e.g., ultrasound, computed tomography (CT) or magnetic resonance imaging (MRI) scans. Tumor size can be determined, for example, by determining tumor weight or tumor volume. As used herein, a reduction of tumor size refers to a rejection of the tumor diameter or tumor volume. The decrease in size can be, for example, a decrease of tumor diameter of 0.01 mm, 0.05 mm, 0.10 mm, 0.12 mm, 0.14 mm, 0.16 mm, 0.18 mm, 0.20 mm, 0.25 mm, 0.30 mm, 0.35 mm, 0.40 mm, 0.45 mm, 0.50 mm, 0.6 mm, 0.7 mm, 0.8 mm, 0.9 mm, 1.0 mm, 1.1 mm, 1.2 mm, 1.3 mm, 1.4 mm, 1.5 mm, 1.75 mm, 2.0 mm, 3.0 mm, 4.0 mm, 5.0 mm, 6.0 mm, 7.0 mm, 8.0 mm, 9.0 mm, 10.0 mm or more. The decrease in size can be a decrease in tumor volume of 10 mm³, 20 mm³, 30 mm³, 40 mm³, 50 mm³, 75 mm³, 100 mm³, 150 mm³, 200 mm³, 250 mm³, 300 mm³, 350 mm³, 400 mm³, 500 mm³, 600 mm³, 700 mm³, 800 mm³, 900 mm³, 1000 mm³ or more. In specific embodiments, such decreases or reductions in tumor size can be, for example, at least a 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% reduction in tumor size. In specific embodiments, treatment or treating encompasses a reduction in the number of tumors in a subject. The decrease in tumor number can be a decrease of at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or more tumors in a subject.

In some embodiments, treatment or treating encompasses a reduction in the spread or the progression of a cancer. The spread or progression of cancer can be reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control. The spread or progression of cancer can be determined by measuring the tumor size, tumor number, tumor location, or any other method known in the art for measuring spread or progression of cancer.

Treating cancer can result in a decrease in number of metastatic lesions in other tissues or organs distant from the primary tumor site. Preferably, after treatment, the number of metastatic lesions is reduced by 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% compared to the number of metastatic lesions prior to administration of a modulator of CXorf67 expression or activity. The number of metastatic lesions may be measured by any reproducible means of measurement. The number of metastatic lesions may be measured by counting metastatic lesions visible to the naked eye or at a specified magnification.

Treating cancer can result in an increase in average survival time of a population of treated subjects in comparison to a population of untreated subjects. In some embodiments, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a modulator of CXorf67 expression or activity. Preferably, the mortality rate is decreased by more than 2%, 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. A decrease in the mortality rate of a population of treated subjects may be measured by any reproducible means. A decrease in the mortality rate of a population may be measured, for example, by calculating for a population the average number of disease-related deaths per unit time following initiation of treatment with a modulator of CXorf67 expression or activity. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% relative to the rate prior to administration of the modulator of CXorf67 expression or activity. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder or cancer can result in a decrease in the number or proportion of cells having an abnormal appearance or morphology. Preferably, after treatment, the number of cells having an abnormal morphology is reduced by at least 5% %, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% relative to the same measurement prior to treatment with a modulator of CXorf67 expression or activity. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

In some embodiments, the subject is characterized by having elevated or increased PRC2 activity when compared to a proper control. As used herein, the term “increased” or “elevated” refers to any increased in the activity of PRC2 when compared to the corresponding activity of PRC2 in a control cell, such as a non-cancerous cell. Such an increase may be up to 5%, 10%, 20%, 30%, 40%, 50%, 60%, 70%, 80%, 90%, 95%, or up to 100%.

In some embodiments, treatment or treating encompasses a reduction in at least one symptom of any disease or condition resulting from the interaction of CXorf67 and PRC2. In specific embodiments, a modulator of CXorf67 expression or activity can reduce the interaction of CXorf67 with PRC2 and thereby treat any condition resulting from the interaction of CXorf67 and PRC2 or any condition in the interaction of CXorf67 and PRC2 is contributing or aggravating factor. These conditions can be identified by measuring the interaction of CXorf67 with PRC2 as disclosed elsewhere herein. The symptom can be reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control.

As described herein, a modulator of CXorf67 expression or activity can be administered in an effective amount in order to treat the cancer or cell proliferative disorder in the subject. In certain embodiments, an “effective amount” or a “therapeutically effective amount” of a modulator of CXorf67 expression or activity can be sufficient to achieve a desired clinical result, including but not limited to, for example, ameliorating disease, stabilizing a subject, preventing or delaying the development of, or progression of, a proliferative disease, disorder, or condition in a subject. In specific embodiments, an effective amount is any amount sufficient to treat cancer or a cell proliferative disorder as described herein. For example, an effective amount is any amount of a modulator or inhibitor of CXorf67 expression or activity sufficient to reduce the tumor size, tumor number, reduce tumor spread, or reduce the progression of a cancer or cell-proliferative disorder. In some embodiments, and effective amount is any amount of a modulator or inhibitor of CXorf67 expression or activity sufficient to modulate or reduce the methylation of of H3K27. An effective amount of therapy can be determined based on one administration or repeated administration. Methods of detection and measurement of the indicators above are known to those of skill in the art. Such methods include, but are not limited to measuring reduction in tumor burden, reduction of tumor size, reduction of tumor volume, reduction in proliferation of secondary tumors, decreased solid tumor vascularization, expression of genes in tumor tissue, presence of biomarkers, lymph node involvement, histologic grade, and nuclear grade. “Positive therapeutic response” refers to, for example, improving the condition of at least one of the symptoms of a cancer, decreasing tumor size or tumor number, and/or reducing the progression of the cancer or cell proliferation disorder.

The specific therapeutically effective dose level for any particular subject will depend upon a variety of factors including the disorder being treated and the severity of the disorder; activity of the specific modulator of CXorf67 expression or activity employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al., (2004), Applied Therapeutics: The Clinical Use of Drugs, Lippincott Williams & Wilkins, ISBN 0781748453; Winter, (2003), Basic Clinical Pharmacokinetics, 4.sup.th ed., Lippincott Williams & Wilkins, ISBN 0781741475; Sharqel, (2004), Applied Biopharmaceutics & Pharmacokinetics, McGraw-Hill/Appleton & Lange, ISBN 0071375503). For example, it is well within the skill of the art to start doses of agents at levels lower than those required to achieve the desired therapeutic effect and to gradually increase the dosage until the desired effect can be achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of compositions described herein can occur as a single event, a periodic event, or over a time course of treatment. For example, agents can be administered daily, weekly, bi-weekly, or monthly. As another example, agents can be administered in multiple treatment sessions, such as 2 weeks on, 2 weeks off, and then repeated twice; or every 3rd day for 3 weeks. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Inhibitory molecules such as, inhibitory small molecules, nucleic acid molecules, such as siRNA or shRNA, ribozymes, peptides, antibodies, antagonist, aptamers, and peptidomimetics that reduces the expression or activity of CXorf67 can be introduced into primary eukaryotic cells using any method known in the art for introduction of molecules into eukaryotic cells. By “introducing” is intended presenting to the eukaryotic cell the expression cassette, mRNA, or polypeptide in such a manner that the sequence gains access to the interior of the primary eukaryotic cell. The methods provided herein do not depend on a particular method for introducing an expression cassette or sequence into a primary eukaryotic cell, only that the polynucleotide or polypeptide gains access to the interior of at least one primary eukaryotic cell. Methods for introducing sequences into eukaryotic cells are known in the art and include, but are not limited to, stable transformation methods, transient transformation methods, and virus-mediated methods.

The modulator or inhibitor of CXorf67 expression or activity as described herein can be administered according to methods described herein in a variety of means known to the art. The modulator or inhibitor of CXorf67 expression or activity can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body. Administration can be parenteral, pulmonary, oral, topical, intradermal, intramuscular, intraperitoneal, intravenous, subcutaneous, intranasal, epidural, ophthalmic, buccal, or rectal administration.

Any modulator or inhibitor of CXorf67 expression or activity as disclosed herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the modulator or inhibitor of CXorf67 expression or activity in a manner similar to that used for delivering insulin or chemotherapy to specific organs or tumors. Typically, using such a system, a modulator or inhibitor of CXorf67 expression or activity can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Modulators or inhibitors of CXorf67 expression or activity can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006), Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

A. Treatment of Cancer

Methods and compositions are provided herein for treating cancer in a subject having cancer by modulating or decreasing the expression or activity of CXorf67. As used herein, “cancer” refers to any cell-proliferative disorder in which unregulated or abnormal growth, or both, of cells can lead to the development of an unwanted condition or disease. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. A cell proliferative disorder includes a non-cancer condition or disorder. Preferably, the methods provided herein are used to treat or alleviate a symptom of cancer. The term “cancer” includes solid tumors, as well as, hematologic tumors, and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

As used herein, a “normal cell” is a cell that cannot be classified as part of a “cell proliferative disorder”, “cancer”, or “tumor”. A normal cell lacks unregulated or abnormal growth, or both, that can lead to the development of an unwanted condition or disease. Preferably, a normal cell possesses normally functioning histone methylation.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders, psoriasis; eczema; atopic dermatitis; hyperpigmentation disorders, eye-related hyperproliferative disorders, age-related macular degeneration, ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalencephaly; rare hypoglycemia, Klippel-Trenaunay syndrome; harmatoma; Cowden syndrome; or overgrowth-hyperglycemia.

Exemplary cancers include, but are not limited to, ependymoma, PFA and other molecular groups of ependymoma, germinoma, adrenocortical carcinoma, AIDS-related cancers, AIDS-related lymphoma, anal cancer, anorectal cancer, cancer of the anal canal, anal squamous cell carcinoma, angiosarcoma, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, head and neck squamous cell carcinoma, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, T-cell lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, lung squamous cell carcinoma, AIDS-related lymphoma, non-Hodgkin lymphoma, primary central nervous system lymphoma, B-cell lymphoma, primary effusion lymphoma, Waldenstram macroglobulinemia, medulloblastoma, melanoma, intraocular (eye) melanoma, merkel cell carcinoma, lewis cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, pancreatic endocrine tumor, paranasal sinus and nasal cavity cancer, parathyroid cancer, cholangiocarcinoma, penile cancer, pharyngeal cancer, pheochromocytoma, pineoblastoma and supratentorial primitive neuroectodermal tumors, pituitary tumor, pituitary adenoma, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

In specific embodiments, the cancer is associated with elevated levels of PRC2 activity. In some embodiments, the cancer is an ependymoma or germinoma. Ependymomas are neuroepithelial tumors of the central nervous system (CNS), presenting in both adults and children but accounting for almost 10% of all pediatric CNS tumors and up to 30% of those in children under 3 years. In children, most ependymomas arise in the posterior fossa, while most adult ependymomas present around the lower spinal cord and spinal nerve roots. Ependymomas can be classified according to each of the three major anatomic compartments in which they are found: supratentorial (ST), posterior fossa (PF), and spinal (SP). In the ST compartment, two molecular groups (ST-EPN-RELA and ST-EPN-YAP1) align with tumors harboring specific genetic alterations, RELA and YAP1 fusion genes. Among PF ependymomas, two of three molecular groups, PFA (PF-EPN-A) and PFB (PF-EPN-B), account for nearly all tumors; PF-SE tumors are rare, generally showing the morphology of a subependymoma. In specific embodiments, the modulator of CXorf67 expression or activity treats a PF ependymoma in a subject. In some embodiments, the modulator of CXorf67 expression or activity treats a PF ependymoma, such as a PFA ependymoma. In specific embodiments, the PFA ependymoma is in a subject under 18 yrs, 16 yrs, 15 yrs, 14 yrs, 13 yrs, 12 yrs, 11 yrs, 10 yrs, 9 yrs, 8 yrs, 7 yrs, 6 yrs, 5 yrs, 4 yrs, 3 yrs, 2 yrs, or under 1 yr old, or 1-5 yrs, 2-4 yrs, or 2-3 yrs old. In some embodiments, the PFA ependymoma occurs in adults, such as adults aged 18-35 yrs, 35-50 yrs, 50-60 yrs, 60-70 yrs, 70-80 yrs, or 80-120 years old.

In some embodiments, the cancer can be a germinoma. As used herein, a germinoma is a germ cell tumor which is not differentiated and can include any malignant neoplasm of the germinal tissue of the gonads, mediastinum, or pineal region. In specific embodiments, the modulator of CXorf67 expression or activity treats an intracranial germinoma, such as an intracranial germinoma at or near the midline, such as in the pineal or suprasellar areas. In some embodiments, the germinoma is in a subject under 18 yrs, 16 yrs, 15 yrs, 14 yrs, 13 yrs, 12 yrs, 11 yrs, 10 yrs, 9 yrs, 8 yrs, 7 yrs, 6 yrs, 5 yrs, 4 yrs, 3 yrs, 2 yrs, or under 1 yr old, or 1-5 yrs, 2-4 yrs, or 2-3 yrs old.

In some embodiments, the present invention provides for a pharmaceutical composition comprising a modulator of CXorf67 expression or activity, as disclosed herein. The modulator of CXorf67 expression or activity can be suitably formulated and introduced into a subject or the environment of the cell by any means recognized for such delivery. Such pharmaceutical compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. In some embodiment a synthetic carrier is used wherein the carrier does not exist in nature. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The pharmaceutical compositions can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

For a compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an T cell or demethylating agent (including, e.g., a protein, polypeptide, or antibody) can include a single treatment or, preferably, can include a series of treatments.

The pharmaceutical compositions can be included in a kit, container, pack, or dispenser together with instructions for administration.

The present invention provides for both prophylactic and therapeutic methods of treating a subject at risk of (or susceptible to) a chronic disease or infection. “Treatment”, or “treating” as used herein, can refer to the application or administration of a therapeutic agent (e.g., modulator of CXorf67 expression or activity) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., a modulator of CXorf67 expression or activity). Subjects at risk for the disease can be identified by, for example, one or a combination of diagnostic or prognostic assays as known in the art. Administration of a prophylactic agent can occur prior to the detection of, e.g., cancer in a subject, or the manifestation of symptoms characteristic of the disease or disorder, such that the disease or disorder is prevented or, alternatively, delayed in its progression.

“Combination therapy” also embraces the administration of the modulator or inhibitor of CXorf67 expression or activity as described herein in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the modulator or inhibitor of CXorf67 expression or activity and non-drug treatment is achieved. For example, in appropriate cases, the beneficial effect is still achieved when the non-drug treatment is temporally removed from the administration of the modulator or inhibitor of CXorf67 expression or activity, perhaps by days or even weeks.

In specific embodiments a modulator or inhibitor of CXorf67 expression or activity can be administered in combination with a chemotherapeutic agent. The chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; an FGFR inhibitor, a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analogue drug or any chemotherapeutic, anti-neoplastic or anti-proliferative agent.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar), In some embodiments, the additional chemotherapeutic agent can be a cytokine such as G-CSF (granulocyte colony stimulating factor).

In particular embodiments, a modulator or inhibitor of CXorf67 expression or activity as disclosed herein can be administered in combination with radiation therapy. Radiation therapy can also be administered in combination with a modulator or inhibitor of CXorf67 expression or activity and another chemotherapeutic agent described herein as part of a multiple agent therapy. In yet another aspect, a modulator or inhibitor of CXorf67 expression or activity, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar) or CMFP (cyclophosphamide, methotrexate, 5-fluorouracil and prednisone).

In particular embodiments, a modulator or inhibitor of CXorf67 expression or activity as disclosed herein can be administered in combination with a modulator or inhibitor of any component or co-factor of the PRC2 complex. For example, a modulator or inhibitor of CXorf67 expression or activity as disclosed herein can be administered in combination with inhibitors of EZH2, SUZ12, and/or EED and/or any other component or co-factor of PRC2 identified herein. Likewise, identification of CXorf67 overexpression in a patient can be followed by treatment of the patient with an inhibitor of PRC2 or a component or co-factor of PRC2 including, but not limited to, of EZH2, SUZ12, and/or EED and/or any other component or co-factor of PRC2 identified herein.

### 4. Methods of Identifying a Patient at Risk of Developing Cancer

Methods and compositions provided herein can identify subjects at an increased risk of developing a cell-proliferative disorder based on a mutation in CXorf67 or increased expression or activity of wildtype CXorf67. In some embodiments, subjects identified as at an increased risk of developing cancer have increased methylation of a histone. For example, subjects identified as at an increased risk of developing cancer have tri-methylated histone H3 at position K27 (i.e., H3K27me3) and in relation to regulatory elements of specific genes. In specific embodiments, a mutation in CXorf67 or overexpression of wildtype CXorf67 indicates that the subject harboring the mutation or overexpression is at an increased risk of developing a cancer. In specific embodiments, overexpression of wildtype CXorf67 indicates that the subject is at an increased risk of developing an ependymoma (e.g., PFA ependymoma) or a germinoma. Likewise, in some embodiments, an increase in CXorf67 expression or an increase in PRC2 activity can indicate that the subject is at an increased risk of developing a cell-proliferative disorder. Based on the assessed risk, a personalized prophylaxis or treatment regimen can be administered to the subject.

As used herein, an “increased risk” of developing a cell-proliferative disorder indicated by a mutation in CXorf67 or increased expression or activity of CXorf67 comprises a statistically significant increase in the risk of developing the cell proliferative disorder. The risk can be based on the presence of a particular risk indicator (e.g., a mutation in CXorf67 polynucleotide) relative to risk in the absence of that risk indicator. The increased risk can include, for example, a risk that is at least about 10% higher, 15% higher, 20% higher, 25% higher, 30% higher, 35% higher, 40% higher, 45% higher, 50% higher, 55% higher, 60% higher, 65% higher, 70% higher, 75% higher, 80% higher, 85% higher, 90% higher, 95% higher, 100% higher, 110% higher, 120% higher, 130% higher, 140% higher, 150% higher, 160% higher, 170% higher, 180% higher, 190% higher, 200% higher, or greater.

In some embodiments, the subject or population of subjects at an increased risk of developing cancer are subjects that have a CXorf67 gene locus that contains at least 5, at least 4, at least 3, at least 2, or at least 1 mutation compared to the wild type CXorf67 sequence. Mutations can be deletions, substitutions, or additions and can occur at any point in the nucleic acid or protein sequence of CXorf67. In some embodiments the mutation occurs at position 30, 71, 73, 79, 81, 88, 93, 105, 110, 113, 114, 116, 122, 157, 184, 214, 228, 249, and/or 366. The mutation in CXorf67 can be S30P, A71T, T73S, I79V, D81Y, D81V, I88F, I88V, L93P, S105I, F110C, V113M, V114G, E116D, E116Q, A122V, A157V, Y184C, R214G, A228V, R249C, and/or A366T. Further mutations in CXorf67 can be identified from the COSMIC and CLINVAR databases. The COSMIC database catalogues somatic mutations in various cancers and can be found at the website cancer.sanger.ac.uk/cosmic. The ClinVar database maintained at NCBI collects information correlating variants of human genes and proteins with disease phenotypes and can be found at the website www.ncbi.nlm.nih.gov/clinvar/. In specific embodiments, the subject at an increased risk harbors a mutation between codon 71 and 122 of the CXorf67 polynucleotide set forth in SEQ ID NO: 1. For example the subject at an increased risk can be a subject with a mutation in at least one of codons 81, 88, or 116 of the CXorf67 polynucleotide.

In particular embodiments, the subject or population of subjects at an increased risk of developing a cell-proliferative disorder, such as cancer exhibit an increased expression of CXorf67 when compared to a proper control. Such an increase in CXorf67 expression that indicates an increased risk of the patient developing a cell-proliferative disorder may be up to 5%, 10%, 20%, 30%, 40%, 50%, 60%, 70%, 80%, 90%, 95%, or up to 100%, 200%, 300%, 400%, or 500%, or more of an increase in CXorf67 expression when compared to an appropriate control. In specific embodiments, an increase in CXorf67 expression indicates that the patient has, or is at risk of developing, an ependymoma or germinoma. In some embodiments, an increase in CXorf67 expression indicates that the patient has, or is at risk of developing, a PFA ependymoma. An increase in the expression of CXorf67 can be measured in any sample taken from the patient, such as a blood or tissue sample, as disclosed elsewhere herein.

Subjects identified as having an increased risk of developing cancer, such as a PFA ependymoma or germinoma, can be administered treatments specific for the individual cancer. Treatments for ependymomas and germinomas include, but are not limited to, surgical resection and radiation, proton therapy, radiotherapy, chemotherapy, administration of TMZ or other alkylating agents, downregulation of ERBB2 and/or ERBB4, and symptom management.

Non-limiting embodiments of the invention include:

1. A method of modifying the activity of polycomb repressive complex 2 (PRC2), said method comprising administering an effective amount of a modulator of CXorf67 expression or activity, wherein modulating the expression or activity of CXorf67 modifies the activity of PRC2.

2. The method of embodiment 1, wherein administering said modulator of CXorf67 expression or activity reduces CXorf67 expression or activity.

3. The method of embodiment 1 or 2, wherein administering said modulator of CXorf67 expression or activity reduces PRC2 activity.

4. The method of any one of embodiments 1-3, wherein administering said modulator of CXorf67 expression or activity reduces methylation at or near the promoter region of CXorf67.

5. The method of any one of embodiments 1-4, wherein administering said modulator of CXorf67 expression or activity reduces methylation of histone H3.

6. The method of embodiment 5, wherein methylation of said histone H3 is reduced at position K27.

7. The method of embodiment 6, wherein methylation of position K27 is reduced from trimethylation status.

8. The method of any one of embodiments 5-7, wherein said histone H3 is located at or near the promoter of a gene of interest.

9. The method of embodiment 8, wherein said gene of interest is CXorf67.

10. The method of any one of embodiments 1-9, wherein histone methyltransferase activity of PRC2 is decreased.

11. The method of any one of embodiments 1-10, further comprising measuring the expression of CXorf67.

12. The method of embodiment 11, comprising measuring overexpression of CXorf67 compared to a proper control.

13. The method of any one of embodiments 1-12, wherein said modulator of CXorf67 is administered to a patient, wherein the level of CXorf67 expression in said patient prior to said administration is increased compared to a control level of CXorf67 expression.

14. The method of embodiment 13, wherein said patient has a PF ependymoma or germinoma.

15. The method of embodiment 14, wherein said PF ependymoma is a PFA ependymoma.

16. The method of any one of embodiments 1-15, wherein administration of an effective amount of said modulator of CXorf67 expression or activity treats or reduces the symptoms of a PFA ependymoma or germinoma following administration to a subject.

17. The method of any one of embodiments 1-16, wherein said administration of an effective amount of a modulator of CXorf67 activity reduces methylation of histone H3

18. A method of identifying a patient at risk of developing a cell-proliferative disorder, said method comprising measuring the expression of CXorf67.

19. The method of embodiment 18, wherein said patient is identified as at risk of developing a cell-proliferative disorder when the level of CXorf67 expression in said patient is increased compared to a control level of CXorf67 expression.

20. The method of embodiment 19, wherein said patient has a PF ependymoma or germinoma.

21. The method of embodiment 20, wherein said PF ependymoma is a PFA ependymoma.

22. The method of any one of embodiments 19-21, further comprising administering an effective amount of a treatment for an ependymoma or germinoma after identifying said patient at risk of developing a cell-proliferative disorder.

23. Use of a modulator of CXorf67 expression or activity in the treatment of cancer or a condition associated with the interaction of CXorf67 and PRC2.

24. Use of a modulator of CXorf67 expression or activity in the manufacture of a medicament for the treatment of cancer or a condition associated with the interaction of CXorf67 and PRC2.

### EXPERIMENTAL

### Example 1. PFA Ependymomas—Recurrent Histone H3 Mutations

We discovered H3 K27M mutations in 13 tumors at a frequency of 4.2%. HIST1H3B (n=5) and HIST1H3C (n=6) were mutated more frequently than H3F3A (n=2). H3 K27M mutations (9/13; 69%) were enriched in PFA-1f, nine mutant tumors representing 39% of tested ependymomas in this minor subgroup. The remaining four tumors occurred at lower frequencies in two other PFA-1 subgroups, PFA-1a (6.4%) and PFA-le (2.5%). Both H3F3A:p.K27M mutations were detected in PFA-1a tumors.

In diffuse midline gliomas, H3 K27M mutations produce widespread reduction of lysine 27 trimethylation (H3 K27me3). Immunohistochemical analysis of a subset of 135 SJ ependymomas showed that this is also true for H3 K27M-mutant PFA ependymomas, but that wild-type PFA ependymomas also display a global loss of H3 K27me3 immunoreactivity, confirming recent results reporting a global loss of H3 K27me3 in PFA ependymomas (Bayliss et al., 2016). The number of H3 mutation-positive cases with clinical data was too low for us to determine reliably, in PFA-1 subgroups, whether tumors with H3 mutations have a poorer outcome than other ependymomas with wild-type H3. However, three of five had progressed within 2 years and died within 4 years.

### Example 2. PFA Ependymomas—Overexpression and Recurrent Mutations in CXorf67

Initial whole genome sequencing studies of ependymoma reported no recurrent SNVs, SVs, or indels in PF tumors (Mack et al., 2014; Parker et al., 2014). Following the discovery of recurrent H3 K27M mutations in our series of ependymomas, we reviewed these original datasets for alterations that could be explored further, finding recurrent mutations in a putative gene, CXorf67, at Xp11.22 on the X chromosome (5 of 30 PF ependymomas; 17. Subsequent targeted sequencing of a subset of PFA tumors (n=234) disclosed a CXorf67 SNV in 22 tumors, at a frequency of 9.4%. CXorf67 missense mutations were found in seven of nine minor subgroups at the following frequencies: PFA-1a 10.3%, PFA-1b 18.8%, PFA-1c 4.2%, PFA-1d 6.3%, PFA-le 9.7%, PFA-2a 6.8%, and PFA-2b 12.5%. CXorf67 and H3 K27M mutations were mutually exclusive, and no ependymoma with a CXorf67 mutation also harbored 1q gain. CXorf67 has one exon, and 15 of 22 mutations (68%) were concentrated in a hotspot region between codons 71 and 122. Three codons in this hotspot had two SNVs each: D81, 188, and E116. The mutant allele was expressed in all tumors.

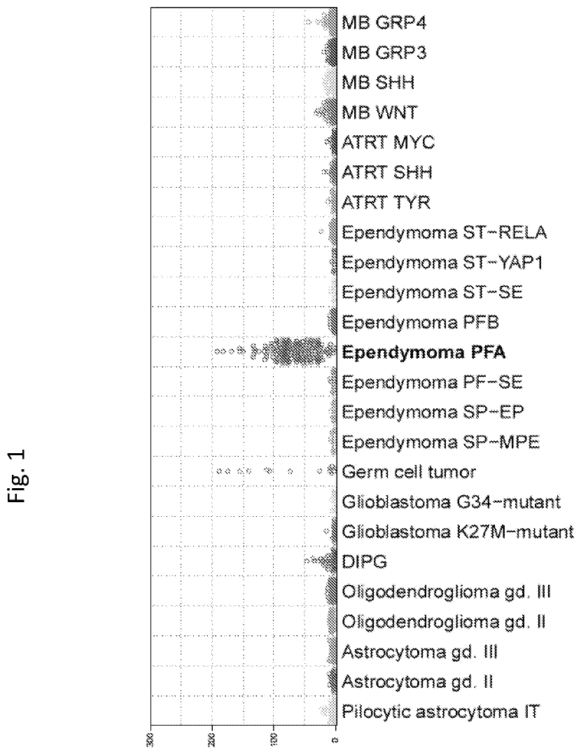

Wild-type CXorf67 is expressed at high levels across PFA ependymomas, but its expression in PFB and ST ependymomas and some other CNS tumors is very low or absent (FIG. 1). The only other tumor in which elevated levels of CXorf67 are consistently found is the germinoma (at both CNS and non-CNS sites). In PFA ependymomas (and germinomas), overexpression is associated with CXorf67 promoter region hypomethylation, in contrast to hypermethylation in other tumor types. There was no apparent difference in the levels of CXorf67 expression in wild-type or mutant PFA tumors.

CXorf67 can be detected at the protein level in tumor cell nuclei by immunohistochemistry and, like H3 K27me3, is a potential biomarker of PFA ependymomas in the formalin-fixed paraffin-embedded (FFPE) tissue samples used for diagnostic purposes. Available tissue sections allowed us to determine that immunoreactivity for the CXorf67 gene product distinguishes PFA from other ependymomas with a sensitivity of 85% and a specificity of 97% (P<0.0001). One exception to this finding is that levels of CXorf67 were noticeably lower, practically immunonegative, in H3-mutant ependymomas than in other PFA tumors.

### Example 3. Significance of Recurrent CXorf67 Mutations Across Distinct Molecular Subgroups of Posterior Fossa Type a (PFA) Ependymoma

DNA methylation profiling previously revealed nine molecular groups, three in each major anatomic compartment (Pajtler et al., 2015). Of those in the posterior fossa (PFA, PFB, and PF-SE), PFA and PFB have been established in several studies as the two principal groups, each with distinct clinicopathologic and biologic associations. PFA ependymomas generally arise in young children and, with a poor outcome, present a major therapeutic challenge. Despite the clinical need, molecular analyses of PFA ependymomas have so far generated few therapeutic leads, and these tumors are essentially treated as they were two decades ago.

The present study aimed to discover molecular heterogeneity of potential clinical and biological relevance among PFA ependymomas. Using DNA methylation profiling, we analyzed a large series of 675 tumors and found two major and nine minor subgroups. We also showed that the two major subgroups, PFA-1 and PFA-2, are distinguished by their gene expression profiles, assignment of an individual tumor to a subgroup aligning precisely across the different methodologies. Some genes differentially expressed in PFA-1 and PFA-2 tumors are involved in CNS patterning during embryogenesis. PFA-1 ependymomas are characterized by high levels of HOX family genes, especially HOXA1-HOXA4 and HOXB1-HOXB4, suggesting a molecular signature related to the development of the caudal brain stem (Alexander et al., 2009). In contrast, PFA-2 tumors demonstrate high levels of genes, such as EN2, CNPY1, IRX3 and OTX2, that are involved in the development of the midbrain/hindbrain boundary or other more rostral posterior fossa sites (Hirate and Okamoto, 2006; Puelles et al., 2003; Sgaier et al., 2007). An analysis of the anatomic relationships and associated radiologic features of a small cohort of SJ tumors, testing the hypothesis that PFA-1 and PFA-2 ependymomas can be distinguished by such metrics, found some significant differences to suggest that the subgroups have distinct origins, but the relevance of such radiologic differences to the results of gene expression profiling awaits further focused study.

Even though PFA-1 and PFA-2 ependymomas showed some differences among their radiologic features, on other clinical metrics there appeared to be very few. In particular, their outcomes were almost identical. In contrast, of the nine minor PFA subgroups, several emerged with distinctive clinical, as well as molecular, characteristics. Age at diagnosis, gender ratio, the ratio of pathologic grades, and outcome as measured by PFS and OS all varied significantly across the minor subgroups. The frequencies at which H3 K27M and Cxorf67 mutations and relatively common CNAs, such as 1q gain and 22q loss, were recorded also differed. Theoretically, DNA methylation profiling can demonstrate heterogeneity down to the level of individual tumors, but an optimum level of granularity for this methodology would deliver molecular subgroups that both have distinctive clinicopathologic, genetic or other biologic characteristics and provide a sound basis for tumor classification, a way of detecting the subgroup in the clinical laboratory, and therapeutic utility.

Contrary to the prevailing view, which asserts that PF ependymomas lack recurrent mutations, we demonstrated that PFA tumors harbor recurrent SNVs in histone H3 genes and a gene of unknown function on the X chromosome, CXorf67. Our data showed that all tumors with H3 K27M mutations were PFA-1 ependymomas, and that two thirds were found in the PFA-1f subgroup. H3 K27M mutations are present in approximately one third of pediatric high-grade gliomas and >70% of diffuse pontine gliomas. This mutation is a hallmark of the diffuse midline glioma, which is incorporated into the WHO classification as a genetically defined entity with a poor prognosis.

Recurrent mutations were found in CXorf67 at a frequency of almost 10% across our series of PFA ependymomas. CXorf67 is a single-exon gene of unknown function, which is located at Xp11.22. The human gene's mRNA contains 1939 bases (orf, 1512 bases), producing a 51.9 kD protein of 503 amino acids. It shows no sequence elements in common with other genes across the human genome. CXorf67 is poorly conserved throughout evolution; the proportions of bases that the human gene shares with those of chimpanzees and mice are 85% and 39%, respectively. Genes that evolve rapidly, showing relatively low levels of sequence similarity across species, are often involved in sexual reproduction (Swanson and Vacquier, 2002), and oocytes, placenta, and testis are the adult tissues in which CXorf67 is expressed. CXorf67 is expressed at high levels during pre-implantation embryonic development, but this has decreased considerably by the blastocyst stage.

Wildtype CXorf67 is expressed at high levels in PFA ependymomas, but not in ependymomas from other molecular groups. It is rarely expressed at high levels in a range of CNS tumors analyzed through the Pediatric Cancer Genome Project (PCGP), and other genomic datasets show that only germinomas among many cancers express CXorf67 at similar levels to those in PFA ependymomas. High levels of CXorf67 in ependymomas and germinomas are associated with promoter region hypomethylation, a phenomenon not seen in multiple other tumor types across the PCGP and The Cancer Genome Atlas (TCGA) datasets.

### Example 4. CXorf67 and PRC2—Functional Interactions of Potential Oncogenic Effect in PFA Ependymomas

While not harboring recurrent mutations at high frequency, PFA ependymomas show widespread epigenetic alterations, including global loss of histone H3 K27-trimethlyation (H3K27-me3) in all cases. Another childhood PF tumor, the diffuse pontine glioma (DPG) also shows global loss of H3K27-me3. In DPGs, loss of H3K27-me3 is associated in most cases with an H3 K27M mutation. Clearly, this mechanism would account for loss of H3K27-me3 in only a small proportion of PFA ependymomas.

PF ependymoma sequencing data at was examined and recurrent mutations were discovered in a novel gene, CXorf67. Targeted sequencing at St. Jude in a series of PFA ependymomas revealed CXorf67 mutations in 22/234 (9.4%). While this is a relatively low frequency of recurrent mutation, it focused our attention on the fact that CXorf67 is highly expressed in >90% of PFA ependymomas.

Mutations in H3 genes and CXorf67 are mutually exclusive across our series of PFA ependymomas and PFA subtypes harbor CXorf67 mutations. Two thirds of the H3 histone mutations (in HIST1H3B, HIST1H3C and H3F3A) are found in PFA-1f ependymomas, among which H3 mutations are present at a frequency of 35%. Thus, wild-type and/or mutated CXorf67 could play a crucial role in PFA ependymomas.

CXorf67 is a single exon gene of unknown function. Its protein product is predicted to be ‘disordered’, apart from one region towards the N terminus. Mutations in PFA ependymomas are missense, and there is a mutation hotspot in the ‘ordered’ region. CXorf67 mutations are not present in other molecular groups of ependymoma and are rare in other cancers, among which there is no evidence for any hotspot region.

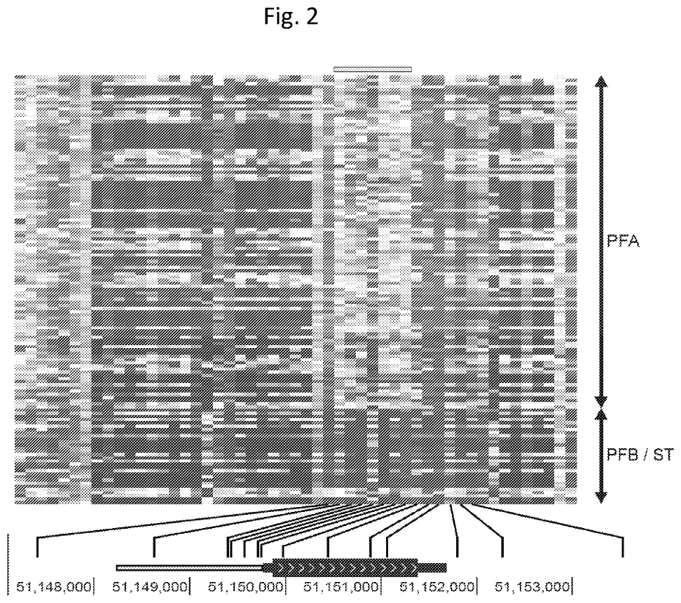

Affymetrix u133v2 arrays were used to establish that CXorf67 is expressed at high levels in PFA ependymomas (PFA-1f tumors being the exception), in contrast to relatively low levels in other ependymomas from the PF and supratentorial compartments. A mechanism for CXorf67 overexpression was revealed in a similar comparative analysis of CpG island methylation profiles, which showed that the promoter region of CXorf67 is hypomethylated in PFA tumors, but not in other ependymomas (FIG. 2). Using immunohistochemical preparations, we detected expression of CXorf67 at the protein level in the nuclei of PFA ependymomas; PFA-1f, PFB and supratentorial tumors were immunonegative. CXorf67 expression is unrelated to mutation status.

Elevated CXorf67 expression is also found in the Daoy and U2-OS cancer cell lines. We used immunoprecipitation (IP)/mass spectrometry (MS) to study proteins bound to CXorf67 in Daoy and U2-OS. Analysis of enriched peptides following immunoprecipitation of CXorf67 indicated that it binds EZH2, SUZ12, and EED, the three core components of the PRC2 complex. Complementary immunoprecipitation of SUZ12 detected CXorf67. CXorf67 has functional effects on H3K27-me3 status, presumably via its interactions with PRC2, which is known to alter the status of H3K27-me3.

### Example 5. CXorf67 Associates with PRC2

Several experiments in cancer cell lines and human neural stem cells (hNSCs) were conducted in order to help understand the relationship between CXorf67 and the components of PRC2. Two cell lines, Daoy and U2-OS, were identified that express CXorf67 at high levels. Using Daoy and U2-OS and an antibody to CXorf67, immunoprecipitation (IP) studies were conducted followed by proteomic analysis/mass spectrometry (MS) or immunoblotting.

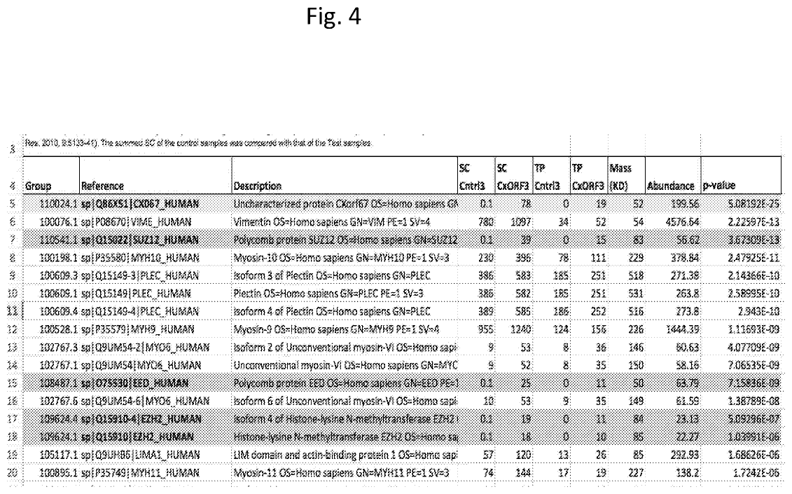

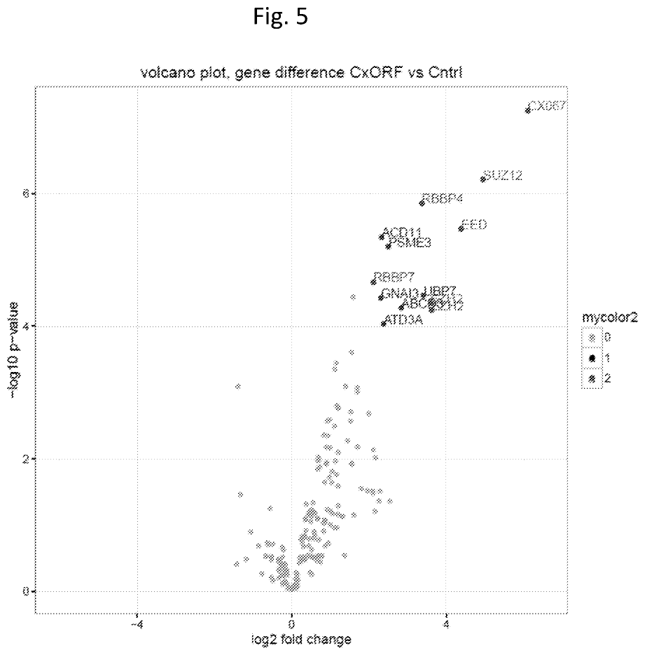

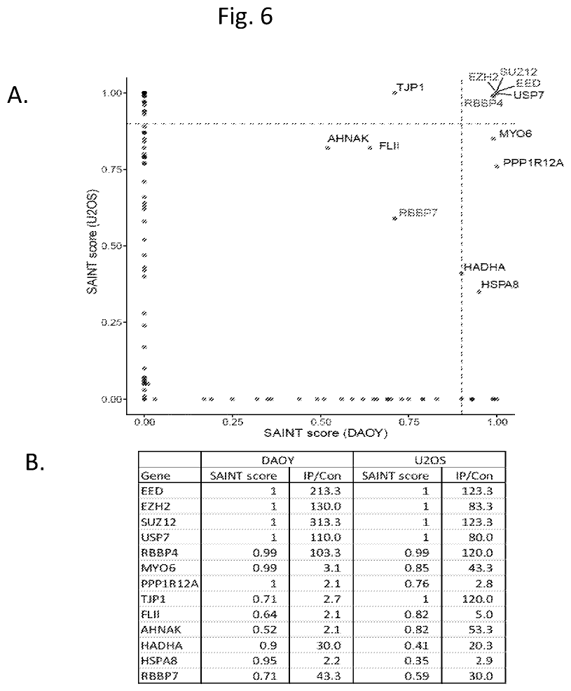

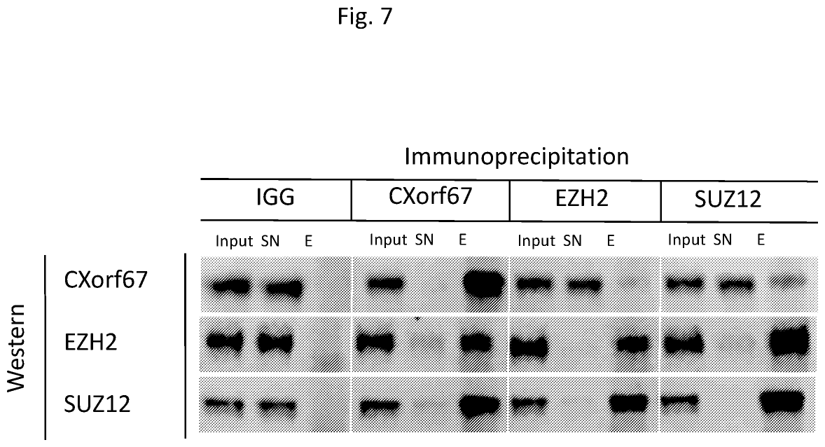

On the basis of the proteomics/MS results obtained, IP with anti-CXorf67 pulls down EZH2, SUZ12, and EED, the three core components of PRC2 (FIG. 4 and FIG. 5). Similar results were found using U2-OS, and data from the two cell lines were combined (FIG. 6). Reciprocal IP-MS data based on pull-down of SUZ12 and EZH2 shows that both PRC2 components bind CXorf67. The immunoblotting data in FIG. 7 demonstrates that a significant amount of CXorf67 remains unbound to EZH2 and SUZ12, two core elements of the PRC2 complex, and a small amount of CXorf67 has been observed in the cytoplasm of cells in PFA ependymomas and Daoy cells. Thus, when the preceding experiments are taken together with the CXorf67 IP-MS data and D3 data, CXorf67 is confirmed to interact with elements of PRC2.

### Example 6. Modulating the Cellular Levels of CXorf67 Alters Levels of H3K27-Me3

In order to confirm that impact of CXorf67 level on histone methylation, several experiments were conducted, including overexpression of CXorf67 in HEK293T cells and hNSCs (in which levels of H3K27-me3 are high) and knockdown of CXorf67 in Daoy cells (in which levels of H3K27-me3 are negligible) using a CRISPR/CAS9 approach.

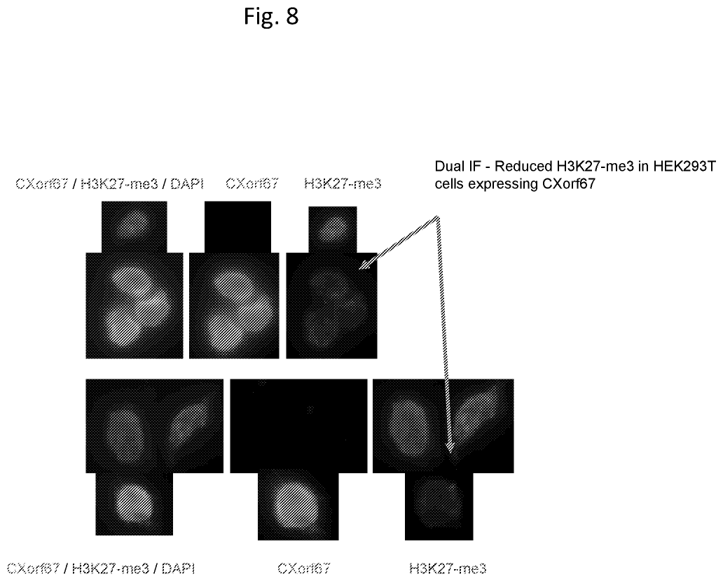

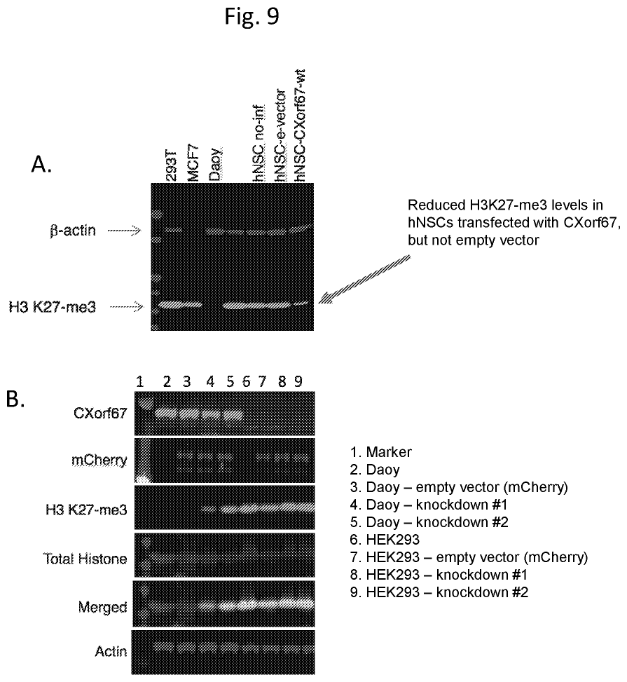

Transient transfection of HEK293T cells produced variable expression of CXorf67, permitting the observation, by dual immunofluorescence, of a reciprocal relationship between levels of CXorf67 and H3K27-me3. See, FIG. 8. In hNSCs, the expression of wildtype CXorf67 produced reduction of H3K27-me3. As presented in FIG. 9, knockdown of CXorf67 in Daoy cells produced an increase in H3K27-me3. Thus, results in these cell lines with divergent levels of CXorf67 and H3K27-me3 support the finding that modulating cellular levels of CXorf67 alters levels of H3 K27-me3.

Overall, these results strongly implicate CXorf67 in the epigenetic dysregulation of PFA ependymomas and that CXorf67 influences H3K27-me3 status in these tumors through an interaction with PRC2. Further, CXorf67 could be a key element of the PRC2 complex.

### Example 7. Modulating Cellular Levels of Murine CXorf67 Alters Levels of H3K7-Me3 in a Mouse Model

In order to investigate whether CXorf67 has the same impact in mouse cells as human CXorf67 has in human cells, a number of experiments were performed. The results confirmed that mouse CXorf67 has the same effect in mouse cells as human CXorf67 has in human cells—a decrease in H3 K27-trimethylation (K27-me³). In view of the effect of CXorf67 on global H3 K27-me³ the function(s) of CXorf67 can be modeled in mouse cells and potentially in genetically engineered mouse models.

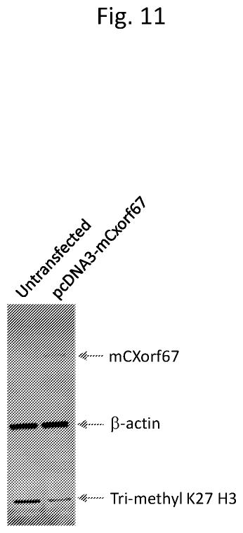

Experiments were performed in mouse NIH3T3 cells, which were demonstrated not to express high levels of CXorf67. A vector was used to to infect the NIH3T3 cells with a construct that would drive the expression of mouse CXorf67. FIG. 11 shows the high level of H3 K27-me³ in normal NIH3T3 cells (untransfected). Infected cells (pcDNA3-mCxorf67) show a significant reduction in H3 K27-me³. An antibody to a FLAG-Tag was used in the construct to show the concomitant expression of CXorf67 given the lack of antibody is availability specific for mouse CXorf67.

1. A method of modifying the activity of polycomb repressive complex 2 (PRC2), said method comprising administering an effective amount of a modulator of CXorf67 expression or activity, wherein modulating the expression or activity of CXorf67 modifies the activity of PRC2.

2. The method of claim 1, wherein administering said modulator of CXorf67 expression or activity reduces CXorf67 expression or activity.

3. The method of claim 1, wherein administering said modulator of CXorf67 expression or activity reduces PRC2 activity.

4. The method of claim 1, wherein administering said modulator of CXorf67 expression or activity reduces methylation at or near the promoter region of CXorf67.

5. The method of claim 1, wherein administering said modulator of CXorf67 expression or activity reduces methylation of histone H3.

6. The method of claim 5, wherein methylation of said histone H3 is reduced at position K27.

7. The method of claim 6, wherein methylation of position K27 is reduced from trimethylation status.

8. The method of claim 5, wherein said histone H3 is located at or near the promoter of a gene of interest.

9. The method of claim 8, wherein said gene of interest is CXorf67.

10. The method of claim 1, wherein histone methyltransferase activity of PRC2 is decreased.

11. The method of claim 1, further comprising measuring the expression of CXorf67.

12. The method of claim 11, comprising measuring overexpression of CXorf67 compared to a proper control.

13. The method of claim 1, wherein said modulator of CXorf67 is administered to a patient, wherein the level of CXorf67 expression in said patient prior to said administration is increased compared to a control level of CXorf67 expression.

14. The method of claim 13, wherein said patient has a PF ependymoma or germinoma.

15. The method of claim 14, wherein said PF ependymoma is a PFA ependymoma.

16. The method of claim 1, wherein administration of an effective amount of said modulator of CXorf67 expression or activity treats or reduces the symptoms of a PFA ependymoma or germinoma following administration to a subject.

17. The method of claim 1, wherein said administration of an effective amount of a modulator of CXorf67 activity reduces methylation of histone H3

18. A method of identifying a patient at risk of developing a cell-proliferative disorder, said method comprising measuring the expression of CXorf67.

19. The method of claim 18, wherein said patient is identified as at risk of developing a cell-proliferative disorder when the level of CXorf67 expression in said patient is increased compared to a control level of CXorf67 expression.

20. The method of claim 19, wherein said patient has a PF ependymoma or germinoma.

21. The method of claim 20, wherein said PF ependymoma is a PFA ependymoma.

22. The method of claim 19, further comprising administering an effective amount of a treatment for an ependymoma or germinoma after identifying said patient at risk of developing a cell-proliferative disorder.

23. Use of a modulator of CXorf67 expression or activity in the treatment of cancer or a condition associated with the interaction of CXorf67 and PRC2.

24. Use of a modulator of CXorf67 expression or activity in the manufacture of a medicament for the treatment of cancer or a condition associated with the interaction of CXorf67 and PRC2.


SEQUENCE LISTING (2 sequences)

SEQ ID NO: 1 - DNA, 1929 bp, Homo sapiens
  misc_feature (1)..(1929): CXorf67 gene sequence
ggactggctgggctccacccaccttcttgctctaccagttcgcgctctcctccggcagaagtagcagctgcgctcttgctctctgggggagaacctggcgttatggccactcagtcagacatggagaaggagcagaagcaccagcaggacgaggggcagggagggctgaacaacgaaaccgcccttgcctccggggatgcctgcgggaccgggaatcaagatcctgctgcttccgtcaccacagtctccagccaagcatctccctcgggcggcgccgccctaagcagcagcacagccggttcttccgctgcagccgccacctccgccgccattttcatcaccgatgaggcctcggggctgccaatcatagctgctgtgctgacggagaggcattctgaccgccaggactgccgcagtcctcacgaagtttttgggtgtgtggtgcccgaggggggcagccaggccgctgtggggccccagaaggccactggccacgccgacgagcacctggcccagaccaagagccccgggaacagccgtcgtaggaagcagccctgccgcaaccaggctgccccggctcagaagcctccagggcggcgtctgtttcctgagcctttgccgccatcttctccagggttccggcccagcagctatccctgttccggggcttctacgtcgagtcaggcaacccagccaggccctgcactcctaagccacgcatctgaggcaaggcctgctacccgaagccgcatcaccctggtagcttctgctctccgcagacgtgcatctggtccaggccctgtcatccgaggctgcaccgcccagccaggccctgcttttccacaccgcgccactcatctagaccctgctcgtctaagccctgaatctgcgccaggccctgcccgccgaggccgtgcatctgtgccaggccctgcccgccgaggctgcgattctgcgccaggccctgcccgccgaggccgcgattctgcgccagtctctgccccccgaggccgcgattctgcgccaggctctgcccgccgaggccgcgattctgcgccaggccctgcccttcgcgtccgcacagcaaggtcagacgccggtcatcgcagcaccagcacgacgccaggcactggcctccggagccgttccacccagcaaagatcagcccttctcagccgccgctctctgtctgggtcagctgatgagaatccttcctgtgggactggctcagaaaggcttgcctttcagagcagatcaggctctcctgatcctgaggtcccaagccgtgcttccccgcctgtttggcatgcagtccgtatgcgtgcctcctcaccctcaccccctgggaggttcttccttcccatccctcagcagtgggatgagagctcctcctcctcctatgcttccaactcctcctccccgagtaggtctcctggcctaagcccctcatccccttcccctgagtttttgggcctgagatctatctccactcctagccctgaaagccttaggtatgctttgatgcctgagttttatgctctgagccctgtccctccagaagagcaggcagaaatagagagcacagctcaccccgcaacaccgcctgagccgtgaccctctataagcatcctctaagacccacagaaggaatcaacatttacccactttgtgaatgggctgcctgccctgtagttagctgcgaggtctcccaaagttccagccgttaaccaacatcctgagtagtttagaagcctctcctccccatgactccaccacctagcacttaacctgctgttggcctttgattctgggccaccttcttcctcaagagtttgcctttgcccttgagctgagatttctgttttccaagttgcttttctgttatgaaatgttttaattagtaaaataaattgttttgtttaaacaaaa

SEQ ID NO: 2 - protein, 503 aa, Homo sapiens
  MISC_FEATURE (1)..(503): CXorf67 protein sequence
MATQSDMEKEQKHQQDEGQGGLNNETALASGDACGTGNQDPAASVTTVSSQASPSGGAALSSSTAGSSAAAATSAAIFITDEASGLPIIAAVLTERHSDRQDCRSPHEVFGCVVPEGGSQAAVGPQKATGHADEHLAQTKSPGNSRRRKQPCRNQAAPAQKPPGRRLFPEPLPPSSPGFRPSSYPCSGASTSSQATQPGPALLSHASEARPATRSRITLVASALRRRASGPGPVIRGCTAQPGPAFPHRATHLDPARLSPESAPGPARRGRASVPGPARRGCDSAPGPARRGRDSAPVSAPRGRDSAPGSARRGRDSAPGPALRVRTARSDAGHRSTSTTPGTGLRSRSTQQRSALLSRRSLSGSADENPSCGTGSERLAFQSRSGSPDPEVPSRASPPVWHAVRMRASSPSPPGRFFLPIPQQWDESSSSSYASNSSSPSRSPGLSPSSPSPEFLGLRSISTPSPESLRYALMPEFYALSPVPPEEQAEIESTAHPATPPEP